(19) United States
(12) Patent Application Publication
(10) Pub. No.: US 2019/0037896 A1
(43) Pub. Date: Feb. 7, 2019

(54) COOLING FORMULATIONS

(71) Applicant: WM. WRIGLEY JR. COMPANY, Chicago, IL (US)

(72) Inventors: Sonya JOHNSON, Chicago, IL (US); Gloria SHELDON, Chicago, IL (US); Minmin TIAN, Chicago, IL (US)

(73) Assignee: WM. WRIGLEY JR. COMPANY, Chicago, IL (US)

(21) Appl. No.: 15/764,712

(22) PCT Filed: Sep. 27, 2016
(86) PCT No.: PCT/US16/53865

§ 371 Date: Mar. 29, 2018

Related U.S. Application Data
(60) Provisional application No. 62/235,380, filed on Sep. 30, 2015.

(51) Int. Cl. A23L 27/20; A23G 3/36; A23G 4/06; C07C 69/75

(52) U.S. Cl. CPC A23L 27/204; A23G 3/36; C07C 2601/14; C07C 69/75; A23V 2002/00; A23G 4/06

(57) ABSTRACT

The present application discloses long-lasting cooling formulations which comprise at least one cooling agent and are suitable for use in confectionery products. In certain, non-limiting embodiments of the disclosed formulations, the cooling agent is WS-30 (glycerol ester of p-menthane-3-carboxylic acid) In certain embodiments, the disclosed formulations provide enhanced cooling attributes for mints and chewing gum.

### FIELD

The presently disclosed subject matter relates to cooling formulations, particularly for use in confectionery products. Specifically, the present disclosure is directed to flavor compositions that include at least one cooling compound. The flavor compositions can enhance a cooling attribute, such as the perceived clean and cool feeling of various delivery system formats, such as mints, gum, and the like.

### BACKGROUND

Confectionery products are known to have a variety of attributes, with cooling attributes of particular interest. Numerous confectionery products are formulated to impart long-lasting cooling attributes with the use of various cooling agents. Such products may include mints or gums, among others.

Efforts have been directed at perfecting the use of cooling agents within chewing gum and other confectionery formulations to enhance flavor composition and control their release to enhance and prolong the flavor of chewing gum or other confectioneries. U.S. Pat. No. 6,627,233 (hereby incorporated herein by reference in its entirety) discloses a number of physiological cooling agents and combinations of physiological cooling agents, including N-2,3-trimethyl-2-isopropyl butanamide (called WS-23), as well as their use in chewing gum.

Cooling, in particular, is a desirable consumer sensory attribute as it is correlated with breath freshening, cleanliness, and the increased overall liking of a product. At present, there is still a need for alternative novel cooling formulations for confectionery products with a high release rate from the products, improved flavor, cooling effects, and overall quality. The presently disclosed subject matter addresses this need as discussed in detail below.

### SUMMARY OF THE INVENTION

The presently disclosed subject matter is directed to a flavor composition comprising WS-30, which chemical structure is as follows:

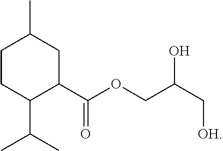 [SMILES: CC1CCC(C(C)C)C(C(=O)OCC(O)CO)C1]

Additionally, the presently disclosed subject matter is directed to a food product comprising the flavor composition.

In certain embodiments, the flavor composition is present at a concentration of from about 0.01 to about 0.5% weight/weight of the food product.

In certain embodiments, the food product is a compressed mint. In certain embodiments, the food product is chewing gum.

In certain embodiments, the flavor composition is present at a concentration of from about 0.01 to about 0.5% weight/weight of the chewing gum.

The foregoing has outlined broadly the features and technical advantages of the present application in order that the detailed description that follows may be better understood. Additional features and advantages of the application will be described hereinafter which form the subject of the claims of the application. It should be appreciated by those skilled in the art that the conception and specific embodiment disclosed may be readily utilized as a basis for modifying or designing other structures for carrying out the same purposes of the present application. It should also be realized by those skilled in the art that such equivalent constructions do not depart from the spirit and scope of the application as set forth in the appended claims. The novel features which are believed to be characteristic of the application, both as to its organization and method of operation, together with further objects and advantages will be better understood from the following description.

### DETAILED DESCRIPTION

As noted above, to date, there remains a need in the art for flavor compositions that provide cooling attributes to various confectionery products. The presently disclosed subject matter addresses this need through the use of at least one cooling compound that provides high cooling intensity, increased duration of cooling, perception of cleanliness, and an increased overall liking and quality of a product.

### 1. Definitions

The terms used in this specification generally have their ordinary meanings in the art, within the context of this disclosed subject matter and in the specific context where each term is used. Certain terms are discussed below, or elsewhere in the specification, to provide additional guidance to the practitioner in describing the compositions and methods of the disclosed subject matter and how to make and use them. As used herein, the use of the word “a” or “an” when used in conjunction with the term “comprising” in the claims and/or the specification may mean “one,” but it is also consistent with the meaning of “one or more,” “at least one,” and “one or more than one.” Still further, the terms “having,” “including,” “containing” and “comprising” are interchangeable and one of skill in the art is cognizant that these terms are open ended terms.

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value.

As used herein, “ppm” means parts-per-million and is a weight relative parameter. A part-per-million is a microgram per gram, such that a component that is present at 10 ppm is present at 10 micrograms of the specific component per 1 gram of the aggregate mixture.

As used herein “admixing,” refers to the process where the cooling formulation is mixed with or added to the completed product or mixed with some or all of the components of the product during product formation or some combination of these steps. When used in the context of admixing, the term “product” refers to the product or any of its components. This admixing step can include a process selected from the step of adding the cooling formulation to the product, spraying the cooling formulation on the product, coating the cooling formulation on the product, suspending the cooling formulation in the product, painting the cooling formulation on the product, pasting the cooling formulation on the product, encapsulating the product with the cooling formulation, mixing the cooling formulation with the product and any combination thereof. The cooling formulation can be a liquid, dry powder, spray, paste, suspension and any combination thereof.

As used herein, the term “chewing gum” refers to a flavored substance intended for chewing. The term as used herein also includes bubble gum and confectionery products containing chewing gum. In certain embodiments, chewing gum forms include, but are not limited to, tablets, sticks, solid balls, hollow balls, cut and wrap, and pellets or pillows. Unless otherwise specified, all percentages used herein are weight percents. As used herein, chewing gum contains a water insoluble base portion and a water-soluble bulk portion.

As used herein, the term “compressed mint” refers to a hard confectionery product that requires sufficient strength to bite and/or shatter and primarily dissolves in the mouth by sucking.

As used herein, “physiological cooling agents” encompasses any number of physiological cooling agents but does not include traditional flavor-derivatives such as menthol or menthone. Preferred physiological agents provide a cooling effect without imparting perceptible flavor of their own. Several known compounds have what can be characterized as a “cooling” activity, and arc referred to in the art as “physiological cooling agents.” Physiological cooling agents arc perceived as cold or cool when contacted with the human body and, in particular, with the mucous membranes of the mouth, nose and throat.

As used herein, the term “Log P value” or the “partition coefficient” is the ratio of the concentration of a specific compound in n-octanol to its concentration in water. In the context of physiological cooling compounds as disclosed herein, a high log P value corresponds to lower water solubility and a decreased ability for coolant release from a product, for example coolant release from a chewing gum composition.

### 2. Cooling Formulations

The present application relates to cooling formulations that include at least one, two, three, or more cooling compounds.

In certain non-limiting embodiments, the cooling formulation comprises the compound WS-30 (glycerol ester of p-menthane-3-carboxylic acid) having the following structure:

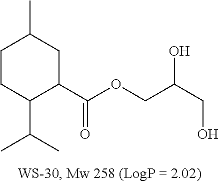 [SMILES: CC1CCC(C(C)C)C(C(=O)OCC(O)CO)C1]

The present disclosure also provides for positional and stereoisomers of the compound WS-30.

In yet other non-limiting embodiments, the cooling formulations can further comprise one, two, three, four, five or more cooling compounds, for example, as described by U.S. Pat. No. 7,025,999; and U.S. Publication Numbers 2007/0248717, 2013/0156885, and 2005/0202118, each of which is incorporated by reference in its entirety herein. In certain embodiments, the secondary cooling compound is WS-23 (N,2,3-Trimethyl-2-isopropylbutamide). In certain embodiments, the secondary cooling compound is WS-5 (N-(Ethoxycarbonylmethyl)-3-p-menthanecarboxamide). In certain embodiments, the secondary cooling compound is WS-12((1R,2S,5R)—N-(4-Methoxyphenyl)-5-methyl-2-(1-methylethyl)cyclohexanecarboxamide). In certain embodiments, the secondary cooling compound is selected from the group consisting of menthyl glutarate, menthyl succinate, isopulegol, menthyl lactate, menthol, WS-3, menthol EG carbonate, menthol PG carbonate, menthol glycerol ether, and combinations thereof.

In one non-limiting embodiment, the cooling formulation comprises WS-30, WS-5, WS-12, WS-23, menthyl glutarate, menthyl succinate, isopulegol, menthyl lactate, menthol, WS-3, menthol EG carbonate, menthol PG carbonate, menthol glycerol ether or combinations thereof.

In one specific non-limiting embodiment, the cooling formulation comprises WS-30, WS-5 and WS-12. In another non-limiting embodiment, the cooling formulation comprises WS-23 and WS-30. In another non-limiting embodiment, the cooling formulation comprises WS-5 and WS-30. In another non-limiting embodiment, the cooling formulation comprises WS-12 and WS-30. In another non-limiting embodiment, the cooling formulation comprises menthyl glutaratc and WS-30. In another non-limiting embodiment, the cooling formulation comprises menthyl succinate and WS-30. In another non-limiting embodiment, the cooling formulation comprises isopulegol and WS-30. In another non-limiting embodiment, the cooling formulation comprises menthyl lactate and WS-30. In another non-limiting embodiment, the cooling formulation comprises menthol and WS-30. In another non-limiting embodiment, the cooling formulation comprises WS-3 and WS-30. In another non-limiting embodiment, the cooling formulation comprises menthol EG carbonate and WS-30. In another non-limiting embodiment, the cooling formulation comprises menthol PG carbonate and WS-30. In another non-limiting embodiment, the cooling formulation comprises menthol glycerol ether and WS-30.

In certain embodiments, the cooling formulations can be used to enhance or increase the cooling and clean feeling attribute of an edible composition, such as chewing gum or compressed mints. In further embodiments, the cooling formulations provide a perceivable intense cooling sensation in the edible food composition. In further embodiments the cooling formulations disclosed herein can provide a consumer perceived decreased menthol sensation in the edible food composition. In certain embodiments, the cooling formulation comprises two or more cooling compounds that provide a greater than additive (i.e., synergistic) enhancement of a cooling sensory attribute when admixed with an edible food composition.

### 3. Food Products

The cooling formulations of the present application can be used to enhance or modify a cooling sensory attribute of various edible compositions such as chewing gum and sweet goods, for example, mints and chewy candies. In one embodiment, such enhancement is an increase in the release, intensity and/or longevity of a cooling sensory attribute that is detectable, for example, by a sensory panelist, as described herein, compared to an edible composition that does not comprise the cooling formulations of the present application. The cooling formulations can include combinations of cooling compounds, and can be added to edible compositions in various delivery system formats.

In certain embodiments of the application, an edible composition can be produced that contains a sufficient amount of at least one, two, three, four, five or more cooling formulation(s), or its various subgenuses described herein, for example, a cooling compound, such as WS-30, to produce an edible composition having the desired cooling sensory attribute.

In certain embodiments, the application relates to methods for enhancing or modulating a cooling sensory attribute of an edible product comprising: a) providing at least one edible food product, or a precursor thereof, and b) combining the edible food product or precursor thereof with at least a cooling sensory attribute modulating amount of at least one, two, three, four, five, or more cooling formulation(s), or any of its subgenuses, for example, one, two three, four, five or more cooling compounds, such WS-30, so as to form a modified edible food product.

In certain embodiments, at least a cooling sensory attribute modulating amount of one, two, three, four, five or more of the cooling formulations can be added to the edible food product, so that the cooling sensory attribute modified edible food product has an enhanced cooling sensory attribute, as compared to the edible food product prepared without the cooling formulation, as determined by human beings or animals in general, or in the case of formulation testing, as determined by a taste panel of at least one, two, three, four, five or more human taste testers, via procedures known in the art.

In certain embodiments of the present application, the cooling formulation is added to an edible food product in an amount effective to enhance a cooling sensory attribute. In one non-limiting example, admixing a cooling formulation comprising WS-30 with an edible food product provides the admixture with an enhanced cooling sensory attribute, and a enhance clean and cool feeling, compared to an admixture comprising an individual cooling compound of the cooling formulation.

The concentration of cooling formulation admixed with an edible food product to modulate or enhance a cooling sensory attribute of the edible food product or composition can vary dependent on variables, such as, for example, the specific type of edible composition, what cooling compounds are already present in the edible food product and the concentrations thereof, and the enhancer effect of the particular cooling formulation on such cooling compounds.

A broad range of concentrations of the cooling formulations can be employed to enhance or modify a cooling sensory attribute of an edible food product. In certain embodiments of the present application, the cooling formulation is admixed with a food product wherein the cooling formulation is present in an amount of from about 1 to about 5000 ppm, or from about 5 to about 4000 ppm, or from about 10 to about 3000 ppm, or from about 50 to about 2000 ppm, or from about 100 to about 1000 ppm, or from about 150 to about 500 ppm, or from about 200 to about 250 ppm, and values in between.

In certain embodiments of the present application, the cooling formulation is admixed with a food product wherein the cooling formulation is present in an amount of from about 0.01 to about 0.54% weight/weight (w/w), or from about 0.1 to about 0.54% w/w, and values in between.

In one non-limiting embodiment, the cooling formulation is admixed with a food product wherein the cooling formulation is present in an amount of about 0.05% w/w. In certain embodiments, said cooling formulation comprises the cooling compound WS-30.

In one non-limiting embodiment, the cooling formulation is admixed with a food product wherein the cooling formulation is present in an amount of about 0.1% w/w. In certain embodiments, said cooling formulation comprises the cooling compound WS-30.

In one non-limiting embodiment, the cooling formulation is admixed with a food product wherein the cooling formulation is present in an amount of about 0.2% w/w. In certain embodiments, said cooling formulation comprises the cooling compound WS-30. In certain embodiments, the cooling formulation is admixed with a food product in an amount effective to enhance a cooling sensory attribute by about 1 to about 10 fold, or from about 1.25 to about 8 fold, or from about 1.5 to about 6 fold, or from about 1.75 to about 4 fold, or from about 2 to about 2.5 fold, and values in between, compared to a cooling sensory attribute reference.

In certain embodiments, the cooling formulations of the present application can be incorporated into a delivery system for use in edible compositions. For example, the cooling formulations can be delivered encapsulated or neat. Delivery systems can be liquid or solid, aqueous or non-aqueous. Delivery systems are generally adapted to suit the needs of the cooling formulation and/or the edible composition into which the cooling formulation will be incorporated.

### 3.1 Confectionery Products

In certain embodiments, cooling formulations of the presently disclosed subject matter can be incorporated into a confectionery product.

In certain embodiments, cooling formulations of the present disclosure can be incorporated into the confectionery product by admixing the inventive formulation into conventional hard and soft confections. In certain embodiments, cooling formulations of the presently disclosed subject matter can be incorporated into confectionery products including, but not limited to, cakes, cookies, pies, candies (hard and soft), compressed mints, chewing gums, chewy candies, gelatins, ice creams, sorbets, jams, jellies, chocolates, fudge, fondant, liquorice, and taffy. In specific embodiments, the cooling formulations of the presently disclosed subject matter are incorporated into compressed mints, chewing gum, soft and chewy candies.

The presently disclosed subject matter can also be incorporated into chewing gum. In certain embodiments, the chewing gum can be in the form of tablets, sticks, solid balls, hollow balls, cut and wrap, and pellets or pillows. Suitable processes for making chewing gum and additional components that may be incorporated into chewing gum are described by way of example in U.S. Pat. No. 8,557,323; U.S. Publication Nos. 2013/0156885 and U.S. 2005/0202118, each of which is incorporated by reference in its entirety herein.

### 3.1.1 Mints

The presently disclosed subject matter can be incorporated into mint confectionaries, and more particularly into compressed mint products using conventional tablet pressing procedures and equipment and suitable additional components known in the art, for example, as described by U.S. Pat. No. 8,557,323 and U.S. Pat. No. 8,431,150, each of which is incorporated by reference in its entirety herein.

### 3.1.2 Chewing Gum

The presently disclosed subject matter can be incorporated into chewing gum using conventional procedures and equipment and suitable additional components known in the art, for example, as described by U.S. Publication Nos. 2013/0156885 and U.S. 2005/0202118, each of which is incorporated by reference in its entirety herein. In certain embodiments, the presently disclosed subject matter is encapsulated before being incorporated into chewing gum.

A chewing gum center composition or other chewing gum compositions can contain a chewable gum base portion, which is essentially free of water and is water-insoluble, a water-soluble bulk portion and flavors which can be water insoluble. The water-soluble portion can dissipate with a portion of the flavor over a period of time during chewing. The gum base portion is retained in the mouth throughout the chew.

In certain embodiments, the insoluble gum base comprises elastomers, elastomer solvents, plasticizers, waxes, emulsifiers and/or inorganic fillers. In certain embodiments, the insoluble gum base can comprise elastomers, elastomer solvents, plasticizers, waxes, emulsifiers and/or inorganic fillers. Plastic polymers, such as polyvinyl acetate, which can behave as plasticizers, can also be included. In certain embodiments, plastic polymers can include but are not limited to, polyvinyl laureate, polyvinyl alcohol and polyvinyl pyrrolidone. Non-limiting examples of elastomers can include polyisobutylene, butyl rubber, (isobutylene-isoprene copolymer) and styrene butadiene rubber, as well as natural latexes such as chicle. In certain embodiments, elastomer solvents can include resins such as terpene resins. In certain embodiments, the plasticizers are fats and oils, including but not limited to, tallow, hydrogenated and partially hydrogenated vegetable oils, and cocoa butter. In certain embodiments, the waxes include, but are not limited to, paraffin, microcrystalline and natural waxes such as beeswax and carnauba.

In certain embodiments, the chewing gum further contains one or more flavor components that are derived from artificial or natural sources or combinations thereof. In certain embodiments, the chewing gum can contain sugar, or may be sugar-free. In certain embodiments, the chewing gum can comprise high potency sweeteners including, but not limited to, synthetic substances, saccharin, thaumatin, alitame, saccharin salts, aspartame, sucralose, stevia, and acesulfame.

In certain embodiments, the chewing gum further contains one more coloring agents. In certain embodiments, the coloring agents can be food quality dyes.

In certain embodiments, the insoluble gum base constitutes between about 5% to about 95% by weight of the gum. In certain embodiments, the insoluble gum base comprises between about 10% and about 50% by weight of the gum or about 20% to about 35% by weight of the gum.

In certain embodiments, the chewing gum contains from about 5% to about 80% bulk sweetener.

In certain embodiments, the high potency sweetener can comprise from about 0.02% to about 1.5%, or from about 0.05% to about 0.0.5% by weight of the chewing gum formulation.

In certain embodiments, the flavor can be added to the chewing gum formula in an amount such that it will contain from about 0.1% to about 10% flavor, from about 0.2% to about 4.0% flavor, or from about 0.5% to about 2% flavor.

In certain embodiments, the cooling formulation includes WS-30 and comprises from about 0.01 to about 0.5% by weight of the chewing gum formulation. In certain embodiments, the cooling formulation includes WS-30 and comprises from about 0.01 to about 0.4% by weight of the chewing gum formulation.

### 3.1.3 Chewy Confectioneries

The presently disclosed subject matter can be incorporated into chewy confectioneries using conventional procedures and equipment and suitable additional components known in the art, for example, as described by U.S. Publication No. 2014/0220188 which is incorporated by reference in its entirety herein.

### EXAMPLES

The presently disclosed subject matter will be better understood by reference to the following Examples, which are provided as exemplary of the disclosed subject matter, and not by way of limitation.

### Example 1

### Chewing Gum

This Example provides formulations of chewing gum containing WS-30 as summarized in Table 1 and analyzes the sensory attributes of each formulation.

TABLE 1
Chewing Gum Formulations

| | Control - A | B | C |
|---|---|---|---|
| Gum base | 25.90% | 25.90% | 25.90% |
| Bulk sweetener | 70.33% | 70.33% | 70.23% |
| Peppermint flavor | 2.12% | 2.12% | 2.12% |
| Softener | 0.8% | 0.8% | 0.8% |
| High potency | 0.75% | 0.75% | 0.75% |
| sweetener | | | |
| WS - 23 | 0.1% | — | — |
| WS - 30 | — | 0.1% | 0.2% |
| TOTAL | 100.00% | 100.00% | 100.00% |

Various sensory attributes were evaluated by an analysis panel. Each attribute was rated on a scale of 1-15 and evaluated at 1 minute, 6 minutes, 12 minutes and post chew as summarized in tables 2-5.

TABLE 2
1 minute metric

| Sensory Attribute | Control - A | B | C |
|---|---|---|---|
| Overall Flavor 1 | 10.3a | 10.2ab | 10.3a |
| Menthol 1 | 3.1a | 2.8b | 3.2a |
| Throat burn 1 | 1.5a | 1.4ab | 1.4ab |
| Tongue burn 1 | 1.5b | 1.5b | 1.9a |
| Icy/Cold | 1.9a | 1.8a | 1.8a |
| Astringent 1 | 1.0b | 1.0ab | 1.0ab |

TABLE 3
6 minute metric

| Sensory Attribute | Control - A | B | C |
|---|---|---|---|
| Overall Flavor 6 | 7.5ab | 7.4ab | 7.7a |
| Menthol 6 | 2.3ab | 2.2ab | 2.4a |
| Throat burn 6 | 1.8a | 1.6a | 1.9a |
| Tongue burn 6 | 1.9a | 1.8a | 1.9a |
| Icy Cold 6 | 1.5a | 1.3b | 1.4ab |
| Astringent 6 | 1.7a | 1.7a | 1.8a |

TABLE 4
12 minute metric

| Sensory Attribute | Control - A | B | C |
|---|---|---|---|
| Overall Flavor 12 | 5.8a | 5.9a | 6.0a |
| Menthol 12 | 1.7b | 1.6b | 1.6b |
| Throat burn 12 | 1.6a | 1.5a | 1.5a |
| Tongue burn 12 | 1.5b | 1.6ab | 1.8ab |
| Icy Cold 12 | 1.3a | 1.3a | 1.3a |
| Astringent 12 | 2.1a | 1.9ab | 1.9b |

TABLE 5
8 minute post chew metric

| Sensory Attribute | Control - A | B | C |
|---|---|---|---|
| Oral Burn -8 | 0.6c | 0.9abc | 0.7c |
| Throat burn -8 | 0.5b | 0.6ab | 0.5ab |
| Tongue burn -8 | 0.5b | 0.6ab | 0.6ab |
| Tongue Numbing -8 | 0.6c | 0.8abc | 0.7bc |
| Icy Cold -8 | 0.6ab | 0.4b | 0.5b |

The chewing gum comprising WS-30 delivered significantly less consumer perceived menthol as compared to the control at 1 minute. Where the lowercase letters in the above tables, e.g., “a”, are different, the particular result was statistically different from the designated sample. Gums B and C containing WS-30 demonstrated similar cooling and other sensory attributes to gum containing WS-23 when evaluated by a trained panel.

Although the presently disclosed subject matter and its advantages have been described in detail, it should be understood that various changes, substitutions and alterations can be made herein without departing from the spirit and scope of the disclosed subject matter as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the presently disclosed subject matter, processes, machines, manufacture, compositions of matter, means, methods, or steps, presently existing or later to be developed that perform substantially the same function or achieve substantially the same result as the corresponding embodiments described herein may be utilized according to the presently disclosed subject matter. Accordingly, the appended claims are intended to include within their scope such processes, machines, manufacture, compositions of matter, means, methods, or steps.

Patents, patent applications publications product descriptions, and protocols are cited throughout this application the disclosures of which arc incorporated herein by reference in their entireties for all purposes.

1. A flavor composition comprising: 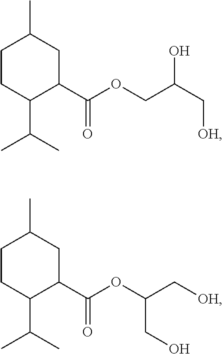 [SMILES: CC1CCC(C(C)C)C(C(=O)OCC(O)CO)C1 ; CC1CCC(C(C)C)C(C(=O)OC(CO)CO)C1]
or combinations thereof.

2. The flavor composition of claim 1, further comprising one or more cooling compounds selected from the group consisting of WS-23 (N,2,3-Trimethyl-2-isopropylbutamide), WS-5 (N-(Ethoxycarbonylmethyl)-3-p-menthanecarboxamide), WS-12 ((1R,2S,5R)—N-(4-Methoxyphenyl)-5-methyl-2-(1-methylethyl)cyclohexanecarboxamide), menthyl glutarate, menthyl succinate, isopulegol, menthyl lactate, menthol, WS-3 (N-Ethyl-p-menthane-3-carboxamide), menthol EG carbonate, menthol PG carbonate, and menthol glycerol ether.

3. The flavor composition of claim 1, further comprising WS-5.

4. The flavor composition of claim 1, further comprising WS-12.

5. The flavor composition of claim 1, further comprising WS-23.

6. The flavor composition of claim 1, further comprising WS-5, WS-12 and WS-23.

7. A food product comprising the flavor composition of claim 1.

8. A food product comprising a flavor composition of: 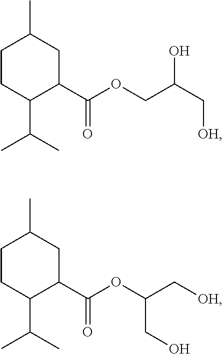 [SMILES: CC1CCC(C(C)C)C(C(=O)OCC(O)CO)C1 ; CC1CCC(C(C)C)C(C(=O)OC(CO)CO)C1]
or combinations thereof, wherein the flavor composition is present at a concentration of from about 0.0001% to about 0.5% weight/weight of the food product.

9. The food product of claim 8, wherein the concentration is about 0.05%, about 0.1% or about 0.2% weight/weight of the food product.

10. The food product of claim 8, wherein the food product is a confectionery product.

11. The food product of claim 8, wherein the food product is selected from the group consisting of cakes, cookies, pies, hard candies, soft candies, compressed mints, chewing gums, chewy candies, gelatins, ice creams, sorbets, jams, jellies, chocolates, fudge, fondant, liquorice, and taffy.

12. The food product of claim 8, wherein the food product is a compressed mint.

13. The food product of claim 8, wherein the food product is chewing gum.

14. The food product of claim 8, wherein the flavor composition comprises WS-30 and comprises from about 0.0001% to about 0.5% by weight of the food product.
